(19) United States
(12) Patent Application Publication
(10) Pub. No.: US 2020/0237367 A1
(43) Pub. Date: Jul. 30, 2020

(54) NOVELTY IN STAPLER EMBODIMENT

(71) Applicant: Turker KARABUGA, Izmir (TR)

(72) Inventor: Turker KARABUGA, Izmir (TR)

(21) Appl. No.: 16/652,578

(22) PCT Filed: Jun. 8, 2018
(86) PCT No.: PCT/TR2018/050295

§ 371 Date: Mar. 31, 2020

(30) Foreign Application Priority Data
Mar. 13, 2018 (TR) 2018/03577

(51) Int. Cl. A61B 17/072; A61F 5/00; A61L 31/02; A61B 17/064

(52) U.S. Cl. CPC A61B 17/072; A61F 5/0083; A61B 2017/07285; A61B 17/064; A61B 2017/07228; A61L 31/022

(57) ABSTRACT

A stapler embodiment has a contact surface in contact with tissue in a surgical operation, an outer wall parallel to the contact surface and in contact with tissue where the surgical operation is conducted for use in surgical operations. The stapler has a left stapling group of more than one staple with the function to staple the tissue where the surgical operation is conducted, in a row position parallel to the contact surface, a right stapling group of more than one staple with the function to staple the tissue where the surgical operation is conducted, in a row position parallel to the outer wall, at least one dissecting embodiment located in the middle of the left stapling group and right stapling group and at least one driving member to provide motion of the left stapling group and right stapling group.

### THE RELATED ART

Invention relates to a stapler embodiment reducing bleeding risk for use in general surgery, particularly sleeve gastrectomy operations.

### BACKGROUND OF THE RELATED ART

Today a surgical tool of dissecting and stapling feature called stapler is used in surgeries of gastrointestinal system. Said surgical tool has two staplers on both sides on vertical axis in current status and blade embodiment is provided in the middle of the stapler appropriate for purpose of dissecting tissue.

However, said stapler is inadequate to ensure safety of anastomosis row in said surgical operations. This causes anastomosis leakage. With the developments made in current applications, number of staplers in stapler embodiment is increased but no improvement has been achieved in anastomosis leakage and symptoms arising from it. In addition, use of said stapler increases bleeding risk and causes some problems during and post operation.

As a result, due to above described drawbacks and deficiencies it has been necessary to make novelty and development in the stapler technology.

### BRIEF DESCRIPTION OF THE INVENTION

The present invention relates to stapler embodiment meeting the needs mentioned above, eliminating all disadvantages and providing some additional advantages.

Main purpose of the invention is to provide a stapler embodiment used in stapling and dissecting operations of tissues in surgical operations conducted in gastrointestinal system and reducing anastomosis leakage risk.

Another purpose of the invention is to disclose a stapler embodiment providing high patient safety by reducing bleeding risk.

In order to achieve the above purposes, the invention relates to a stapler embodiment comprising a contact surface in contact with tissue in surgical operation, an outer wall parallel to said contact surface and in contact with tissue where surgical operation is conducted, for use in surgical operations and it is characterized in comprising left stapling group comprising of more than one staple with function to staple the tissue where surgical operation is conducted, in a row position parallel to said contact surface, right stapling group comprising of more than one staple with function to staple the tissue where surgical operation is conducted, in a position parallel to said outer wall, at least a dissecting embodiment located in the middle of said left stapling group and right stapling group and at least a driving member to provide motion of said left stapling group and said right stapling group.

The structural and characteristics features of the invention and all advantages will be understood better in detailed descriptions with the figures given below and with reference to the figures, and therefore, the assessment should be made taking into account the said figures and detailed explanations.

### BRIEF DESCRIPTION OF FIGURES

FIG. 1 shows view of stapler embodiment disclosed under the invention.

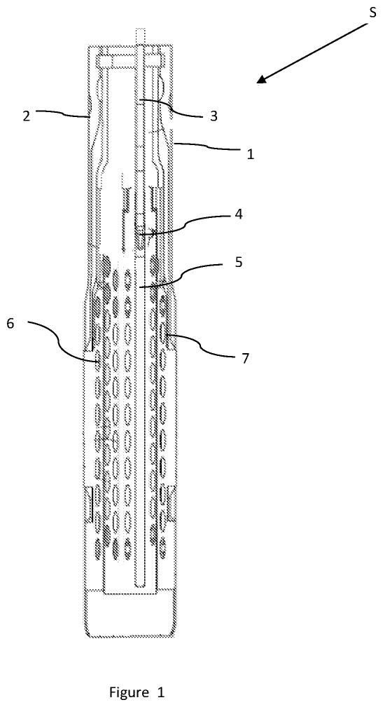

### REFERENCE NUMBERS

S Stapler Embodiment
1 Outer Wall
2 Contact Surface
3 Driving Member
4 Dissecting embodiment
5 Dissecting embodiment housing
6 Right Stapling Group
7 Left Stapling Group

### DETAILED DESCRIPTION OF THE INVENTION

In this detailed description, the preferred embodiments of the invention have been described in a manner not forming any restrictive effect but only for purpose of better understanding of the invention.

Stapler embodiment (S) disclosed under the invention is a surgical tool used to perform dissecting and stapling operations of tissues in surgical operations. Said stapler embodiment (S) has a contact surface (2) in contact with the tissue where surgical operation is conducted.

Stapler embodiment (S) comprising a left stapling group (7) (on left side of said stapler embodiment (S)) in 4 rows suitable for stapling the tissue where surgical operation is conducted and in a position parallel to said contact surface (2), a right stapling group (6) comprising of 2 pieces of staples in a position parallel to said outer wall (1) located on right side of stapler embodiment (S). A dissecting embodiment (4) to perform dissection of tissue is located between the left stapling group (7) located on said contact surface (2) side and the right stapling group (6) located on said outer wall (1) side. Said dissecting embodiment (4) is in blade form and included in dissecting embodiment housing (5). The component providing pushing and thus motion of said left stapling group (7) and the right stapling group (6) is the driving member (3).

1. A stapler embodiment comprising a contact surface in contact with tissue in surgical operation, an outer wall parallel to said contact surface and in contact with tissue where the surgical operation is conducted, for use in surgical operations and it is characterized in comprising:
a left stapling group comprising of more than one staple with function to staple the tissue where the surgical operation is conducted, in a row position parallel to said contact surface;
a right stapling group comprising of more than one staple with function to staple the tissue where the surgical operation is conducted, in a position parallel to said outer wall;
at least one dissecting embodiment located in the middle of said left stapling group and right stapling group; and
at least a one driving member to provide motion of said left stapling group and said right stapling group.

2. The stapler embodiment according to claim 1, and it is characterized in that it comprises a staple made of titanium.

3. The stapler embodiment according to claim 1, and it is characterized in that it comprises of a stapling group comprising of 4 rows of staple.

4. The stapler embodiment according to claim 1, said right stapling group comprising of 2 rows of staple.

5. The stapler embodiment according to claim 1, wherein said dissecting embodiment is in blade form.

6. The stapler embodiment according to claim 1, further comprising a dissecting embodiment housing hosting said dissecting embodiment.
